(19) United States
(12) Patent Application Publication
(10) Pub. No.: US 2026/0137320 A1
(43) Pub. Date: May 21, 2026

(54) A DEVICE FOR MEASURING HANDGRIP STRENGTH AND ASSESSING MUSCLE GROUPS OF VARIOUS BODY PARTS

(71) Applicant: ASSISTAID HEALTHCARE PRIVATE LIMITED, Bengalaru (IN)

(72) Inventors: Siddharth Goel, Bengalaru (IN); Paurvi Kedia, Bengalaru (IN); Shovan Saha, Manipal (IN)

(21) Appl. No.: 19/118,334

(22) PCT Filed: Sep. 30, 2023
(86) PCT No.: PCT/IB2023/059826

§ 371 Date: Apr. 3, 2025

(30) Foreign Application Priority Data
Oct. 3, 2022 (IN) 202241056796

(51) Int. Cl. A61B 5/22; A61B 5/00; G16H 50/30

(52) U.S. Cl. CPC A61B 5/225; A61B 5/7225; G16H 50/30; A61B 2560/0223; A61B 2562/0252; A61B 2562/04

(57) ABSTRACT

The present disclosure relates to a device (100) measuring muscle strength and assessing muscle groups of various body parts. The device (100) incorporates one or more load cells (104-1, 104-2, 104-3) enclosed by a housing 102. The housing (102) adapted to provide gripping of hand and application of force by the user. The one or more load cells (104-1, 104-2, 104-3) generates an electrical signal corresponding to the applied force. The device (100) incorporates an amplifier (106) to amplify the electrical signal and send it to an ADC module (108) to convert it into a digital signal. The ADC module (108) is connected to a computing unit (110), which processes the digital signal and provides data of force applied by the user. The device (100) incorporates a display module (114) to display the measured force.

### TECHNICAL FIELD

The present disclosure relates to the field of measuring devices. More particularly, the present disclosure relates to a device for measuring muscle strength and assessing muscle groups of various body parts.

### BACKGROUND

Background description includes information that may be useful in understanding the present invention. It is not an admission that any of the information provided herein is prior art or relevant to the presently claimed invention, or that any publication specifically or implicitly referenced is prior art.

The structure of a human body is composed of many different types of cells that together create tissues, muscles and subsequently organ systems, which ensures homeostasis and the viability of the human body. The human body comprises various body parts such as a head, neck, trunk (which includes the thorax and abdomen), arms and hands, legs and feet. Physiology focuses on the organs of the human body and their functions. Once any of the organs goes damaged, through a period of recovery and rehabilitation, it becomes important to know if the person's gripping ability and muscle functionality has been restored or not. Otherwise, the person can again experience serious unrecoverable damage to his/her organs, if not recovered completely.

Doctors or physiotherapist or examiners performs various tests/methods on the recovered and rehabilitated body parts of the person to know if his/her body parts such as handgrip, neck, back, leg, arm, foot and finger strength is again ready to perform at least the normal day to day work.

Currently, in the market, we have two main devices. The Jamar Smart+ digital handgrip dynamometer which measures handgrip and costs roughly 45000 INR. The device is imported from US and there is no service available for the same in India. The other device is the Lafayette Dynamometer which costs a minimum of 1.4 Lakhs INR which measures neck, back, leg, arm etc. A professional will have to buy two devices to and still might not be able to measure finger strength. In addition, the two devices can cost them an almost upwards of 2 lakhs INR. Along with that, since these devices are not available in India, these devices are usually imported from outside and for servicing, needs to be sent outside which is expensive, time consuming and in overall, a big task for the professional due to which either professionals tend not to invest these devices at all or purchase these devices but don't service them for years which leads to incorrect readings.

There is therefore need in the art to develop a light weight, accurate, cost effective and single device for a device for measuring muscle strength and assessing muscle groups of various body parts of a user, and provide transfer of the measured data to various digital platforms for storage and analysis.

### OBJECTS OF THE PRESENT DISCLOSURE

Some of the objects of the present disclosure, which at least one embodiment herein satisfies are as listed herein below.

It is an object of the present disclosure to provide a device for measuring muscle strength and assessing muscle groups of various body parts.

It is an object of the present disclosure to provide a single device for measuring hangrip, finger strength, back, neck, foot, arm, leg strength.

It is an object of the present disclosure to provide a device for measuring hangrip, and various muscles related to finger, back, neck, foot, arm, leg strength, which is light weight, accurate and reliable.

It is an object of the present disclosure to provide a single device for measuring different parts of the body, which can calibrate itself.

It is an object of the present disclosure to provide a single device for measuring different parts of the body, which provides digital representation of results with improved resolution and accuracy.

It is an object of the present disclosure to provide a single device for measuring different parts of the body, which can transfer the measured data to various digital platforms for storage and analysis purpose.

### SUMMARY

The present disclosure relates to the field of measuring devices. More particularly, the present disclosure relates to a device for measuring muscle strength and assessing muscle groups of various body parts.

An aspect of the present disclosure pertains to a device for measuring handgrip strength and assessing muscle groups of a user. The device comprising a housing, one or more load cells, an amplifier, analog to digital converter (ADC) module, and a computing unit. The housing adapted to gripped in a hand of a user. The one or more load cells are enclosed inside the housing, the housing configured to translate a force applied on the housing to the one or more load cells, where the one or more load cells are configured to generate a first set of electrical signals corresponding to the force applied on the housing. The amplifier can be operatively coupled to the one or more load cells, the amplifier configured to amplify amplitude of the first set of electrical signals to a predefined amplitude. The analog to digital converter (ADC) module is operatively coupled to the amplifier, the ADC module configured to receive the amplified first set of electrical signals and convert it into a first set of digital signals. The computing unit can be operatively coupled to the ADC module, the computing unit comprising one or more processors coupled with a memory. The memory storing instructions executable by the one or more processors to receive the first set of digital signals from the ADC module and process the received first set of digital signals to generate a second set of digital signals. The second set of digital signals corresponds to the force applied on the housing of the device, where the generated second set of digital signals is processed by the computing unit so as to be represented on at least one mobile computing device.

In an aspect, the device may comprise the one or more load cells are a bending load cells, and the housing configured to translate the force applied on the housing by the user, into a bending force, to the bending load cells.

In an aspect, the device may comprise the one or more load cells comprising a first load cell, a second load cell and a third load cell, the first load cell and the second load cell can be configured to support a predefined weight with an individual capacity, where the predefined weight is 1 kilogram force to 70 kilograms force. The third load cell 104-3 can be configured to support a predefined weight pertaining to a finger of the user, where the predefined weight is 1 kilogram force to 10 kilograms force.

In an aspect, the device can assess one or more muscle groups of the user based on a perpendicular position, an open position and a closed position of a set of handles at a predefined angle. The set of handles can measure one or more movements of a neck, a back, an arm and a hand, where the predefined angle of the perpendicular position, the open position and the closed position pertains to 0 degree, 90 degree and 180 degrees, respectively.

In an aspect, the device can used in one or more posture of the user, wherein the one or more posture comprises at least one of a supine lying posture, a side lying posture, a prone lying posture, a sitting posture and a standing posture.

In an aspect, he one or more load cells comprises one or more resistors configured to change resistance based on the bending force of the device.

In an aspect, the device comprises a communication module operatively which is coupled to the computing unit. The communication module is configured to communicatively couple the device to the at least one mobile computing device and transmit the second set of digital signals corresponding to the force applied on the housing to the at least one mobile computing devices of users.

In an aspect, the device comprises a display module can be operatively coupled to the computing unit, and the generated set of second signals corresponding to the force applied on the housing of the device is processed by the computing unit so as to be represented on the display module.

In an aspect, the device is configured as self-calibrated once the device is turned ON.

In an aspect, the ADC module can be a HX711 ADC convertor, and wherein the computing unit is any or a combination of a Microcontroller, Microprocessor and an Arduino chipset.

Various objects, features, aspects and advantages of the inventive subject matter will become more apparent from the following detailed description of preferred embodiments, along with the accompanying drawing figures in which like numerals represent like components.

Within the scope of this application it is expressly envisaged that the various aspects, embodiments, examples and alternatives set out in the preceding paragraphs, in the claims and/or in the following description and drawings, and in particular the individual features thereof, may be taken independently or in any combination. Features described in connection with one embodiment are applicable to all embodiments, unless such features are incompatible.

### BRIEF DESCRIPTION OF DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure. The diagrams are for illustration only, which thus is not a limitation of the present disclosure.

FIG. 1 illustrates functional modules of the proposed device for a device for measuring muscle strength and assessing muscle groups of various body parts, in accordance with an exemplary embodiment of the present disclosure, to elaborate upon its working.

FIG. 2 illustrates exemplary system architecture of the proposed device and digital platforms, in accordance with an embodiment of the present disclosure.

FIG. 3 illustrates exemplary network architecture in which or with which proposed device and mobile computing devices can be implemented, in accordance with an embodiment of the present disclosure.

FIGS. 4A-4C illustrates architecture the proposed device 100 representing a front view, a rear view and a bottom view of the device 100, in accordance with an embodiment of the present disclosure.

FIGS. 5A-5B illustrates architecture the proposed device 100 representing handle 500 of the device 100, in accordance with an embodiment of the present disclosure.

FIGS. 6A-6B illustrates architecture the proposed device 100 representing switch 600 of the device 100, where FIG. 6A represents power switch button and FIG. 6B represents reset switch button in accordance with an embodiment of the present disclosure.

FIG. 7 illustrates architecture the proposed device 100 representing a display cover 700 of the device 100, in accordance with an embodiment of the present disclosure.

FIG. 8 illustrates architecture the proposed device 100 representing a finger flap 800 of the device 100, in accordance with an embodiment of the present disclosure.

FIG. 9 illustrates architecture the proposed device 100 representing a silicon grip 900 of the device 100, in accordance with an embodiment of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

In the following description, numerous specific details are set forth in order to provide a thorough understanding of embodiments of the present invention. It will be apparent to one skilled in the art that embodiments of the present invention may be practiced without some of these specific details.

If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

Embodiments of the present invention may be provided as a computer program product, which may include a machine-readable storage medium tangibly embodying thereon instructions, which may be used to program a computer (or other electronic devices) to perform a process. The machine-readable medium may include, but is not limited to, fixed (hard) drives, magnetic tape, floppy diskettes, optical disks, compact disc read-only memories (CD-ROMs), and magneto-optical disks, semiconductor memories, such as ROMs, PROMs, random access memories (RAMs), programmable read-only memories (PROMs), erasable PROMs (EPROMs), electrically erasable PROMs (EEPROMs), flash memory, magnetic or optical cards, or other type of media/machine-readable medium suitable for storing electronic instructions (e.g., computer programming code, such as software or firmware).

Exemplary embodiments will now be described more fully hereinafter with reference to the accompanying drawings, in which exemplary embodiments are shown. This invention may, however, be embodied in many different forms and should not be construed as limited to the embodiments set forth herein. These embodiments are provided so that this disclosure will be thorough and complete and will fully convey the scope of the invention to those of ordinary skill in the art. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

The present disclosure relates to the field of measuring devices. More particularly, the present disclosure relates to a device for measuring muscle strength and assessing muscle groups of various body parts.

An aspect of the present disclosure pertains to a device for measuring handgrip strength and assessing muscle groups of a user. The device comprising a housing, one or more load cells, an amplifier, analog to digital converter (ADC) module, and a computing unit. The housing adapted to gripped in a hand of a user. The one or more load cells are enclosed inside the housing, the housing configured to translate a force applied on the housing to the one or more load cells, where the one or more load cells are configured to generate a first set of electrical signals corresponding to the force applied on the housing. The amplifier can be operatively coupled to the one or more load cells, the amplifier configured to amplify amplitude of the first set of electrical signals to a predefined amplitude. The analog to digital converter (ADC) module is operatively coupled to the amplifier, the ADC module configured to receive the amplified first set of electrical signals and convert it into a first set of digital signals. The computing unit can be operatively coupled to the ADC module, the computing unit comprising one or more processors coupled with a memory. The memory storing instructions executable by the one or more processors to receive the first set of digital signals from the ADC module and process the received first set of digital signals to generate a second set of digital signals. The second set of digital signals corresponds to the force applied on the housing of the device, where the generated second set of digital signals is processed by the computing unit so as to be represented on at least one mobile computing device.

In an aspect, the device may comprise the one or more load cells are a bending load cells, and the housing configured to translate the force applied on the housing by the user, into a bending force, to the bending load cells.

In an aspect, the device may comprise the one or more load cells comprising a first load cell, a second load cell and a third load cell, the first load cell and the second load cell can be configured to support a predefined weight with an individual capacity, where the predefined weight is 1 kilogram force to 70 kilograms force. The third load cell 104-3 can be configured to support a predefined weight pertaining to a finger of the user, where the predefined weight is 1 kilogram force to 10 kilograms force.

In an aspect, the device can assess one or more muscle groups of the user based on a perpendicular position, an open position and a closed position of a set of handles at a predefined angle. The set of handles can measure one or more movements of a neck, a back, an arm and a hand, where the predefined angle of the perpendicular position, the open position and the closed position pertains to 0 degree, 90 degree and 180 degrees, respectively.

In an aspect, the device can used in one or more posture of the user, wherein the one or more posture comprises at least one of a supine lying posture, a side lying posture, a prone lying posture, a sitting posture and a standing posture.

In an aspect, the one or more load cells comprises one or more resistors configured to change resistance based on the bending force of the device.

In an aspect, the device comprises a communication module operatively which is coupled to the computing unit. The communication module is configured to communicatively couple the device to the at least one mobile computing device and transmit the second set of digital signals corresponding to the force applied on the housing to the at least one mobile computing devices of users.

In an aspect, the device comprises a display module can be operatively coupled to the computing unit, and the generated set of second signals corresponding to the force applied on the housing of the device is processed by the computing unit so as to be represented on the display module.

In an aspect, the device is configured as self-calibrated once the device is turned ON.

In an aspect, the ADC module can be a HX711 ADC convertor, and wherein the computing unit is any or a combination of a Microcontroller, Microprocessor and an Arduino chipset.

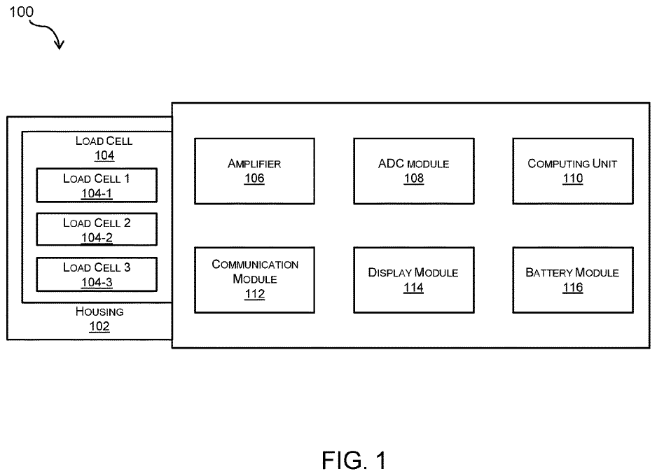

FIG. 1 illustrates functional modules of the proposed device for a device for measuring muscle strength and assessing muscle groups of various body parts, in accordance with an exemplary embodiment of the present disclosure, to elaborate upon its working.

As illustrated, in an embodiment, the proposed device 100 (also known as digimuscle dynamometer 100, herein) for measuring muscle strength and assessing muscle groups of various body parts of a user can include one or more load cells 104 enclosed inside a housing 102 such that the housing 102 can be adapted to be held in hand or gripped by hand of the user to apply force on the device 100. The housing 102 can be configured to translate the force applied on the housing 102, to the load cell 104. Further, the load cell 104 can be configured to generate a first set of electrical signal corresponding to the force applied on the housing 102 by the user.

In an embodiment, the one or more load cells 104 can be a bending load cell 104 and the housing 102 can be configured to translate the force applied on the housing 102 by the hand of the user into a bending force to the bending load cell 104. The one or more load cells 104 comprises a first load cell 104-1 and a second load cell 104-2 configured to support a predefined weight with an individual capacity, where the predefined weight is 1 kilogram force to 70 kilograms force. A third load cell 104-3 is configured to support a predefined weight pertaining to a finger of the user, where the predefined weight is 1 kilogram force to 10 kilograms force. For instance, the load cells 104-1, 104-2 have an individual capacity of 70 kgs, which together provide a total capacity of measurement of 120 kg force. The load cells 104-3 have a capacity of 10 kg which is integrated specially for the fingers. The load cell 104 can include one or more resistors, which change their resistance based on how much it is bent by the applied force. The one or more resistors can form a wheat-stone bridge. When the user grips the device in his hand or applies force on the device with his hand, the imbalance in the wheat-stone bridge can generate a resistance, which generates a low amplitude electrical signal (i.e. a low amplitude first set of first signals) from an excitation voltage provided to the wheat-stone bridge.

In an embodiment, the device 100 is configured to assess one or more muscle groups of the user based on an open position and a closed position of a set of handles at a predefined angle. The set of handles 204 measures one or more movements of a neck, a back, an arm and a hand, where the predefined angle of the open position and the closed position pertains to 90 degree and 180 degrees, respectively. The device 100 is used in one or more posture of the user, where the one or more posture comprises at least one of a supine lying posture, a side lying posture, a prone lying posture, and a standing posture.

In an embodiment, the device 100 can include an analog to digital converter (ADC) module (also referred to as ADC module 108, herein) operatively coupled to the amplifier 106. The ADC module 108 can be configured to receive the amplified first set of electrical signals from the amplifier 106 and convert it into a first set of digital signals.

In an embodiment, the ADC module 108 can be a HX711 ADC module, but not limited to the likes. In a non-limiting embodiment, the HX711 ADC module can include an input multiplexer, which can select either Channel A or B differential input to the low-noise programmable gain amplifier 106 (PGA). Channel A can be programmed with a gain of 128 or 64, corresponding to a full-scale differential input voltage of ±20 mV or ±40 mV respectively, when a 5V supply is connected to a VDD analog power supply pin. Channel B has a fixed gain of 32. The HX711 ADC module can include an on-chip power supply regulator, which can eliminate the need for an external supply regulator to provide analog power for the ADC. The clock input of the HX711 ADC module can be flexible and can be from an external clock source, a crystal, or the on-chip oscillator that does not require any external component.

In an embodiment, the device 100 can include a computing unit 110 operatively coupled to the ADC module 108. The computing unit 110 can include one or more processors coupled with a memory, the memory storing instructions executable by the one or more processors to receive the first set of digital signals from the ADC module 108 and process the received first set of digital signals to generate a second set of digital signals corresponding to the force applied on the housing 102 by the user.

In an exemplary embodiment, the computing unit 110 can selected from any or a combination of a Microcontroller, Microprocessor, Arduino chipset, but not limited to the likes.

In an embodiment, the computing unit 110 can be configured to self-calibrate the device 100 upon turning ON the device and/or whenever the device 100 is used. The device 100 can be kept idle on a flat surface for the calibration process to happen correctly and to ensure removal of errors by zero weight, thereby improving the accuracy of the device. The self or automatic calibration performed by the device can be based on either continuous or periodic calibration of the amplifier 106, ADC module 108, computing unit 110 and/or load cell 104 against two stable references. Before any measurement is done or when the device 100 is turned ON, the computing unit 110 can provide a low reference electrical signal, followed by a high reference electrical signal to the amplifier 106 to finally measure the output value by the computing unit 110. With these two measurements, the offset and the gain can be determined. The actual measurement that follows this can be influenced by the same offset and gain. Now the deviations of the device can be known, and the reading of the actual measurement by the device 100 can be corrected for improved accuracy and reliability.

In an embodiment, the device 100 can include a communication module 112 operatively coupled to the computing unit 110. The communication module 112 can be configured to communicatively couple the device 100 to mobile computing devices 202, and transmit the second set of digital signals corresponding to the force applied on the housing 102 to the mobile computing devices 202.

In an exemplary embodiment, the communication module 112 can be any or a combination of a Bluetooth module, a WIFI module, an IR module, but not limited to the likes.

In an embodiment, the device 100 can include a display module 114 operatively coupled to the computing unit 110 to provide instructions to the user and/or display readings of the measured handgrip force. In an exemplary embodiment, the display module 114 can be or a combination of LED, LCD, PicoLED, AMOLED, but not limited to the likes.

In an embodiment, the computing unit 110 can be configured to process the generated set of second signals corresponding to the force applied on the housing 102 of the device 100, and convert it into a format being displayable on the mobile computing devices and the display module 114 of the device 100.

In an embodiment, the device 100 can include a battery module 116 to provide electrical power to any or a combination of the amplifier 106, ADC module 108, computing unit 110, communication module 112 and display module 114 of the device 100. The battery module 116 can include a set of batteries such as but not limited to rechargeable Li-ion battery, and a battery-charging unit to facilitate charging of the set of batteries. The battery module 116 can further include a power management and safety unit which can be configured to manage transfer of electrical power to any or a combination of the amplifier 106, ADC module 108, computing unit 110, communication module 112 and display module 114 of the device 100, and prevent any mishaps such as but not limited to short circuiting, overcharge, dead charge etc.

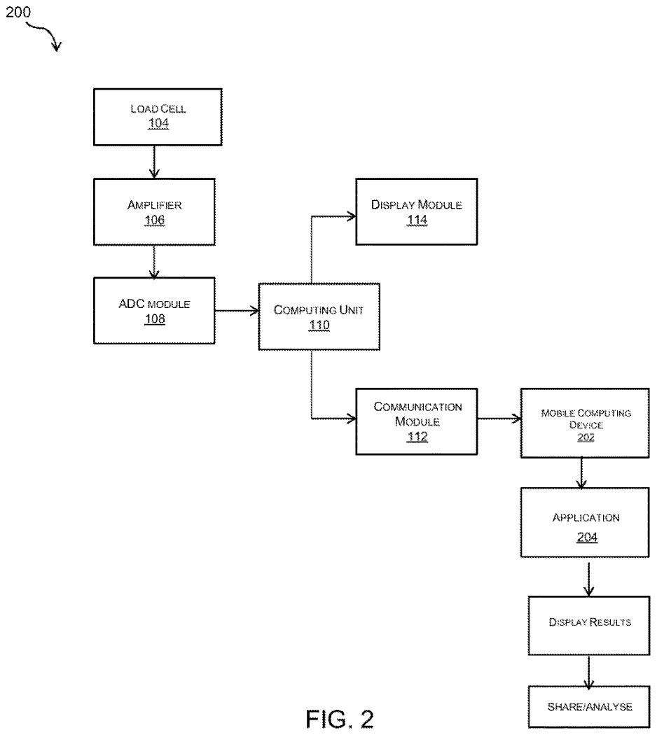

FIG. 2 illustrates exemplary system architecture of the proposed device and digital platforms, in accordance with an embodiment of the present disclosure.

As illustrated, in an embodiment, the exemplary system architecture of the proposed device and digital platforms can include the proposed device 100 being communicatively coupled to mobile computing device 202 through a communication module 112 of the device 100, to provide various digital platforms using mobile applications, computer software and website, etc.

In an embodiment, a user can apply a force on the housing 102 of the device 100. The housing 102 can be configured to transfer the applied force to the one or more load cells 104 of the device 100. The one or more load cells 104 can then generate a first set of electrical signals corresponding to the force applied on the housing 102. The one or more load cells 104 can be operatively coupled to an amplifier 106, which can amplify the amplitude of the first set of electrical signals to a predefined amplitude. The amplified first set of electrical signals having the predefined amplitude can then be send to an ADC module 108 of the device 100. The ADC module 108 can be configured to receive the amplified first set of electrical signals and convert it into a first set of digital signals. The ADC module 108 can be further operatively coupled to a Microcontroller 110. The Microcontroller 110 (also known as computing device) can be configured to receive the first set of digital signals from the ADC module 108 and process the received first set of digital signals to generate a second set of digital signals corresponding to the force applied on the housing 102 of the device 100.

In an embodiment, the generated second set of digital signals corresponding to the force applied on the housing 102 of the device 100 can be processed by the microcontroller 110 to be represented on an on-board display module 114 of the device 100.

In an embodiment, the generated second set of digital signals corresponding to the force applied on the housing 102 can be processed by the microcontroller 110 to be represented on the mobile computing device 202. The device 100 can include a communication module 112 such as a Bluetooth Module, operatively coupled to the microcontroller 110 to establish connection between the proposed device 100 and the mobile computing device 202 such as smart phone, laptop, computer, cloud-based server. The mobile computing device(s) 202 can provide the digital platform for displaying and analyzing the second set of digital signals corresponding to the force applied on the housing 102 of the device, to users, examiners, physiotherapists, doctors, but not limited to the likes.

In an embodiment, an application 204 installed on the mobile computing devices 202 can process the received second set of digital signals corresponding to the force applied on the housing 102 of the device 100 and generates graphs, chart and multiple analysis reports corresponding to the force measured by the proposed device 100. The application 204 can include Android and IOS based mobile application, computer software installed on computer, laptop and a website, but not limited to the likes.

In an embodiment, the application 204 installed on the mobile computing device 202 can process the second set of digital signals corresponding to the force applied on the housing 102 of the device 100 and generate different reports such as average strength, average power, peak power etc. applied by the user. The readings can also be used to generate graphs, which can facilitate an examiner or physiotherapist to understand hand muscle and strength of the user. The graphs can provide a better understanding of the user's strength because such graphs and the multiple reports can show the sustainability of the user's strength to the examiner or physiotherapist to understand the user better.

In a non-limiting embodiment, the device 100 can be configured to provide peak readings for a second or 10 seconds, 20 seconds or more. The examiner can see just the peak reading and judge whether the user requires further treatment or so but the user's inability to hold that value may cause him troubles in his daily work, which is not good. The application 204 installed on the mobile computing device 202 can generate all the graphs and readings and compile it to a PDF report, which can be shared with the user for the user's reference. Along with this, the app (application) can also hold the users or examiner's details, which can be mentioned on the PDF report as a standard header and footer for all PDF reports being shared with the user.

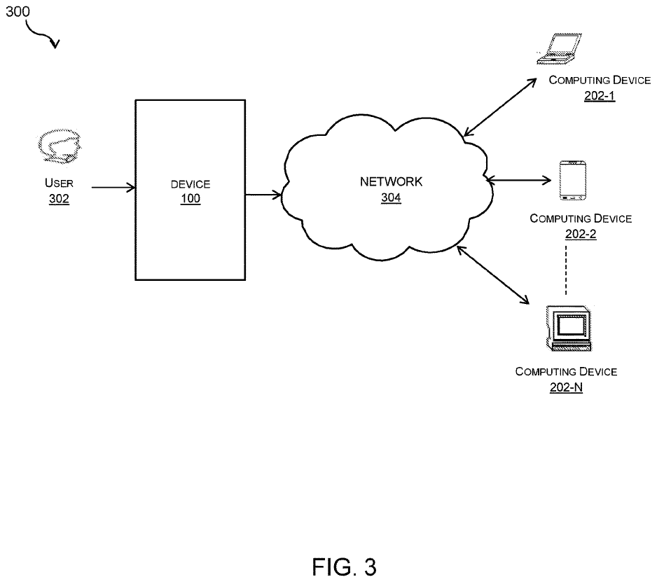

FIG. 3 illustrates exemplary network architecture in which or with which proposed device and mobile computing devices 202 can be implemented, in accordance with an embodiment of the present disclosure.

As illustrated, in an embodiment, the device 100 can be adapted to be held in hand or gripped by hand of a user 302 to apply force on an housing of the device 100. Further, the device 100 can be configured to generate a second set of digital signal corresponding to the force applied by the user on the device 100. The device 100 can include a communication module 112 to connect the device 100 to the mobile computing devices 202 such as a smart phone 202-1, a computer/laptop 202-2 and a cloud based server 202-3. The device 100 can be configured to process the second set of digital signal corresponding to the force applied by the user on the device 100 and transmit it to the mobile computing devices 202 through a network 302.

In an embodiment, the device 100 can be accessed using the mobile computing devices 202 connected to the device. The device 100 can be calibrated and configured using the applications installed on the mobile computing devices 202.

The network architecture in which or with which proposed device and mobile computing devices 202 can be implemented is described demonstrating a single user and a single device. It is to be appreciated that the present disclosure is not limited to single user or a single device being connected to the mobile computing devices 202. However, a plurality of users and their devices can be communicatively coupled to the mobile computing devices 202, and the mobile computing devices 202 can configured to display the readings and report associated with the force being measured by the proposed device.

The user 302 can interact with the mobile computing devices 202 through the network 302 using an application, a web browser, a proprietary program, or any other program executed and operated by the access device. In some embodiments, the access device can communicate directly with the network devices (e.g., communication signal). For example, the access device can communicate directly with network devices using ZigBee™ signals, Bluetooth™ signals, Wi-Fi™ signals, infrared (IR) signals, UWB signals, Wi-Fi-Direct signals, BLE signals, sound frequency signals, or the like. In some embodiments, the access device can communicate with the network devices via the gateways and/or a cloud network.

The local area network can include a wireless network, a wired network, or a combination of a wired and wireless network. A wireless network can include any wireless interface or combination of wireless interfaces (e.g., ZigBee™, Bluetooth™, Wi-Fi™, IR, UWB, Wi-Fi-Direct, BLE, cellular, Long-Term Evolution (LTE), WiMAX™, or the like). A wired network can include any wired interface (e.g., fiber, Ethernet, powerline, Ethernet over coaxial cable, digital signal line (DSL), or the like). The wired and/or wireless networks can be implemented using various routers, access points, bridges, gateways, or the like, to connect devices in the local area network. For example, the local area network can include gateway and gateway. Gateway can provide communication capabilities to network devices and/or access device via radio signals in order to provide communication, location, and/or other services to the devices. The gateway is directly connected to the external network and can provide other gateways and devices in the local area network with access to the external network. The gateway can be designated as a primary gateway.

The network access provided by gateway can be of any type of network familiar to those skilled in the art that can support data communications using any of a variety of commercially-available protocols. For example, gateways can provide wireless communication capabilities for the local area network 100 using particular communications protocols, such as Wi-FiTM (e.g., IEEE 802.11 family standards, or other wireless communication technologies, or any combination thereof). Using the communications protocol(s), the gateways can provide radio frequencies on which wireless enabled devices in the local area network can communicate. A gateway can also be referred to as a base station, an access point, Node B, Evolved Node B (eNodeB), access point base station, a Femtocell, home base station, home Node B, home eNodeB, or the like.

Gateways can include a router, a modem, a range extending device, and/or any other device that provides network access among one or more computing devices and/or external networks. For example, gateway can include a router or access point or a range extending device. Examples of range extending devices can include a wireless range extender, a wireless repeater, or the like.

A router gateway can include access point and router functionality, and can further include an Ethernet switch and/or a modem. For example, a router gateway can receive and forward data packets among different networks. When a data packet is received, the router gateway can read identification information (e.g., a media access control (MAC) address) in the packet to determine the intended destination for the packet. The router gateway can then access information in a routing table or routing policy, and can direct the packet to the next network or device in the transmission path of the packet. The data packet can be forwarded from one gateway to another through the computer networks until the packet is received at the intended destination.

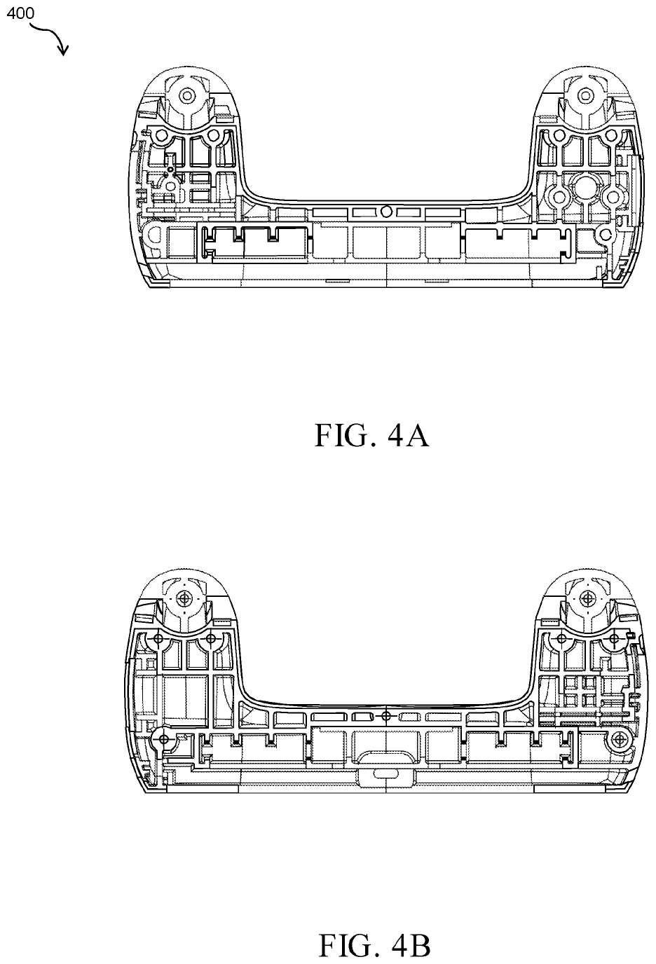

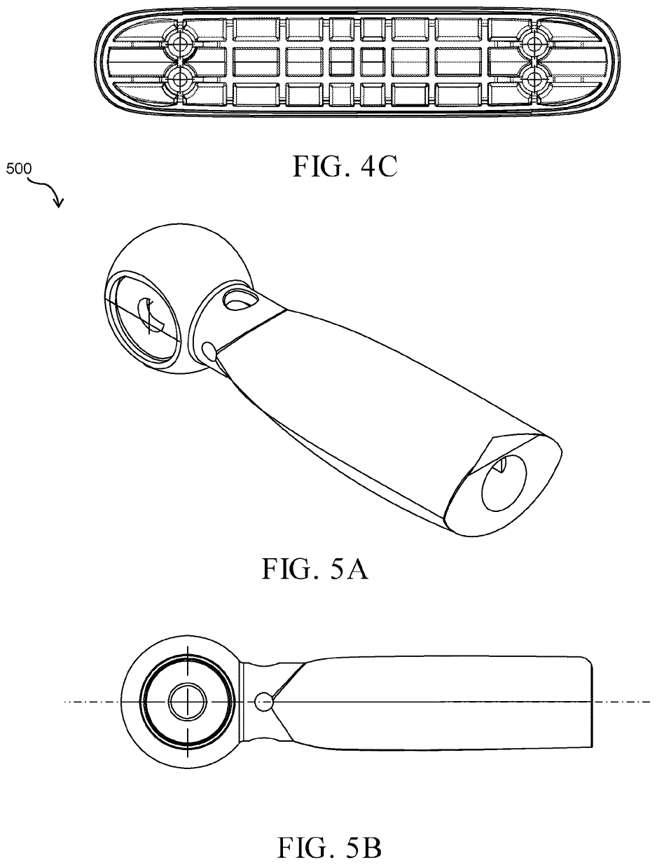

FIGS. 4A-4C illustrates architecture the proposed device 100 representing a front view, a rear view and a bottom view of the device 100, in accordance with an embodiment of the present disclosure.

FIGS. 5A-5B illustrates architecture the proposed device 100 representing handle 500 of the device 100, in accordance with an embodiment of the present disclosure.

In an embodiment, the handle 500 of the device 100 typically refers to the part of the device that you hold or grip while weighing an object. The handle 500 is designed to provide a comfortable and secure grip, allowing the user to manipulate the device 100 easily.

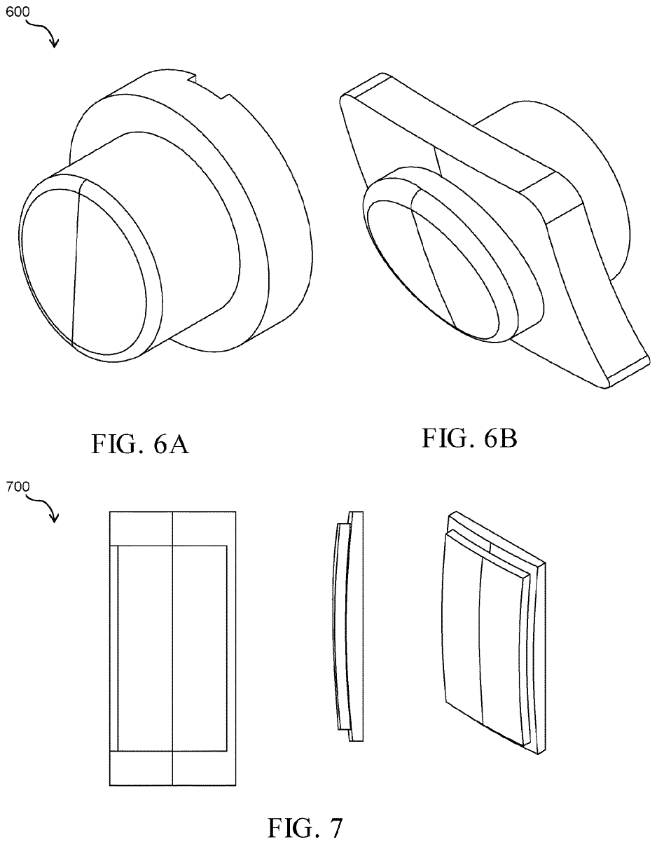

FIGS. 6A-6B illustrates architecture the proposed device 100 representing switch 600 of the device 100, in accordance with an embodiment of the present disclosure.

In an embodiment, the proposed device 100 can include a switch 600, which can be configured to turn on/turn off the device 100. Upon switching it on i.e, power on by using a power switch button FIG. 6A, a tare factor and calibration factor calibrates the device 100 to be used every time it's switched on. Once the device shows a reading of zero, the device can be used to take the reading. FIG. 6B represents reset swtich button which allows the users to reset the device 100 or restore it to its default settings.

FIG. 7 illustrates architecture the proposed device 100 representing a display cover 700 of the device 100, in accordance with an embodiment of the present disclosure.

In an embodiment, the readings of the device 100 are displayed on the display unit 700, which is a 0.97-inch display area configured on the housing 102. The peak strength is displayed on the device 100.

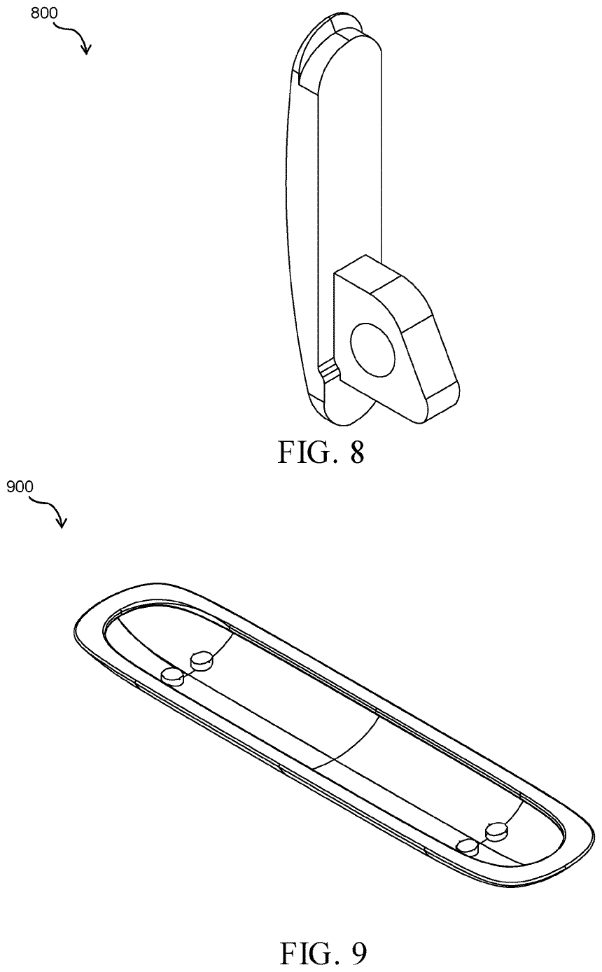

FIG. 8 illustrates architecture the proposed device 100 representing a finger flap 800 of the device 100, in accordance with an embodiment of the present disclosure.

In an embodiment, the finger flap 800 can be extending outwards when it is used, and unfolded back into the housing 102. The finger flap 800 can be configured to measure different types of finger strength, by holding the device 100 in any orientation. When the finger flap 800 is used for measurement, the load cell 104-3 is activated automatically.

FIG. 9 illustrates architecture the proposed device 100 representing a silicon grip 900 of the device 100, in accordance with an embodiment of the present disclosure.

In an embodiment, the silicon grip 900 is used in the finger flap 800, the silicon grip 900 refers to a type of grip or covering made from silicone material. silicone is a synthetic polymer that is known for its flexibility, durability, and heat resistance. the silicon grip 900 can be used in various applications due to its non-slip properties and ability to provide a comfortable grip.

In an embodiment, the proposed device 100 is composed of three major sections for measuring muscle strength and assessing muscle groups of various body parts. First section for measuring hand grip strength, the second section relates to measuring finger and toe strength, and third section for measuring neck muscles, back muscles, arm and forearm muscles, hand muscles, hip muscles, knee and foot muscles.

In an embodiment, the proposed device 100 measures strength of all the muscles collectively that contributes to the grip of the user. There are two methods to measure handgrip strength using the proposed device 100. The first method includes smaller sized hands, and second method includes larger sized hand. The first method depicts for smaller sized hands, where the user one can wrap around the hand to the main cylindrical body of the housing 102 and open the set of handles 500 apart and not use them. The device 100 is placed at the arm side, elbow bent at 90 degrees and forearm in neutral rotation. The device 100 is pressed against the arm, and the peak reading is displayed on the display unit 700 of the device 100.

In another embodiment, the second method depicts for larger sized hands, where the user can wrap around the hand across the entire device 100 including the set of handles 500. The device 100 is placed at the arm side, elbow bent at 90 degrees and forearm in neutral rotation. The device 100 is pressed against the arm, and the peak reading is displayed on the display unit 700 of the device 100. The handgrip is measured by using the load cell 104-1, the load cell 104-2 of 70 kg capacity each to bring the total measurement capacity to 120 kgf.

In an embodiment, the second section of the device 100 for measuring finger and toe strength. The device 100 measures strength of all the finger and toe movements like Metacarpophalangeal Flexion, Extension, Adduction and Abduction. Proximal interphalangeal flexion and extension and Distal interphalangeal flexion and extension. For the thumb the device 100 is capable of measuring MCP flexion extension adduction and abduction. Interphalangeal flexion and abduction For the lower limb this particular part can measure flexion, extension, adduction and abduction of all the 5 digits. During measuring fingers the device 100 uses the load cell 104-3, which has 10 kg and is active only once the finger flap 800 is open. The rest of the load cells get deactivated at this point so one can easily hold the device from anywhere, no matter the orientation as the reading will be taken only from the finger strength measuring metal clip.

In an embodiment, the third section of the device 100 for measuring rest of the body. The third section of the device 100 is capable of measuring all movements of neck, back, arm, hand thighs, legs feet. In the forearm the only strength it cannot measure is the pronation and supination. The device 100 can be operated with a single hand, bimanually (using both hands). By opening and closing the set of handles 500 at 90 degree and 180 degrees, respectively. The device 100 has the capability via its load cell technology to measure 120 kgf of force produced by these muscles. The starting and testing position for each and every muscle can differ. The device 100 can be used in supine lying, side lying, prone lying position. Sitting, high sitting and even standing position is supported by the device. The test position depends upon the personal preference and experience of the person testing the device. All muscles can be tested but are not limited to be used as according to the manual muscle testing techniques used by doctors and rehabilitation professionals.

In another embodiment, the device 100 is placed distal to the muscle being tested on the body of the person being tested. The person testing the strength places the device 100 on the part to be tested and instructs the person being tested to try to move the part in the particular direction against the force applied by through the device by the person testing the strength. The force that the person produced in a particular muscle to move the joint is recorded as the muscle strength of that particular muscles. For example: to measure the strength of biceps. The examinee would sit on a chair with arms by the side and elbow flexed at 90 degrees. The examiner will then place the device at the region proximal to the wrist and ask the examinee to flex the elbow further with as much power as possible. Now the examiner will apply a resistance force in downwards direction through the device. The strength produced by the biceps of the examinee in an attempt to flex the elbow is the recording shown on the device 100, which indicates the peak strength of the biceps. The readings are further divided as an average, time to peak and a graph on the application end of the system and gives further clarity and insight in the working of the muscle of the examinee.

In an embodiment, the device 100 can be configured for measuring muscle strength and assessing muscle groups of various body parts obtained by executing a particular movement by the device 100. For instance, if a particular part of the thumb of the user has to be accessed for example the base of the thumb. Thumb CMC (Carpometacarpal) joint refers to the joint at the base of the thumb, where the thumb meets the wrist. CMC joint adduction specifically refers to the movement of the thumb towards the palm or inwards towards the other fingers to measure muscle strength and assesses muscle groups.

In an embodiment, the device 100 can be configured to conduct one or more test which includes but not limited to a Shoulder Flexion, a Shoulder Extension, a Shoulder Abduction, a Shoulder Adduction, a Shoulder Internal rotation, a Shoulder External rotation, a Elbow Flexion, a Elbow Extension, a Radio-Ulnar Joint Pronation, a Radio-Ulnar Joint Supination, a Wrist Flexion, a Wrist Extension, a Hand Grip, a Thumb CMC Joint Flexion, Thumb CMC Joint Extension, a Thumb CMC Joint Abduction, a Thumb CMC Joint Adduction, a Thumb MCP Joint Flexion, a Thumb MCP Joint Extension, a Thumb MCP Joint Abduction, a Thumb MCP Joint Adduction, a Thumb IP Joint Flexion, a Thumb IP Joint Extension, an Index Finger MCP Joint Flexion, an Index Finger MCP Joint Extension, an Index Finger MCP Joint Abduction, an Index Finger MCP Joint Adduction, an Index Finger PIP Joint Flexion, an Index Finger PIP Joint Extension, an Index Finger DIP Joint Flexion, an Index Finger DIP Joint Extension, a Middle Finger MCP Joint Flexion, a Middle Finger MCP Joint Extension, a Middle Finger MCP Joint Abduction, a Middle Finger MCP Joint Adduction, a Middle Finger PIP Joint Flexion, a Middle Finger PIP Joint Extension, a Middle Finger DIP Joint Flexion, a Middle Finger DIP Joint Extension, a Ring Finger MCP Joint Flexion, a Ring Finger MCP Joint Extension, a Ring Finger MCP Joint Abduction, a Ring Finger MCP Joint Adduction, a Ring Finger PIP Joint Flexion, a Ring Finger PIP Joint Extension, a Ring Finger DIP Joint Flexion, a Ring Finger DIP Joint Extension, a Little Finger MCP Joint Flexion, a Little Finger MCP Joint Extension, a Little Finger MCP Joint Abduction, a Little Finger MCP Joint Adduction, a Little Finger PIP Joint Flexion, a Little Finger PIP Joint Extension, a Little Finger DIP Joint Flexion, a Little Finger DIP Joint Extension, a Neck Flexion, a Neck Extension, a Neck Lateral Flexion L, a Neck Lateral Flexion R, a Neck Rotation to R, a Neck Rotation to L, a Back Flexion, a Back Extension, a Back Lateral Flexion L, a Back Lateral Flexion R, a Back Rotation to R, a Back Rotation to L, a Hip Flexion, a Hip Extension, a Hip Abduction, a Hip Adduction, a Hip Internal rotation, a Hip External rotation, a Knee Flexion, a Knee Extension, an Ankle Plantar Flexion, an Ankle DorsiFlexion, an Ankle Inversion, an Ankle Eversion, and the likes.

In an embodiment, the device 100 can also be connected to one or more computing device 202 such as smartphone via bluetooth using an ESP32 module to generate graphs, progress reports and even muscle charts, which act as added functionality when connected to the smartphone application.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### ADVANTAGES OF THE INVENTION

The proposed disclosure provides a device for measuring muscle strength and assessing muscle groups of various body parts.

The proposed disclosure provides a single device for measuring hangrip, and various muscles related to finger, back, neck, foot, arm, leg strength.

The proposed disclosure provides a device for measuring hangrip, finger strength, back, neck, foot, arm, leg strength, which is light weight, accurate and reliable.

The proposed disclosure provides a single device for measuring different parts of the body, which can calibrate itself.

The proposed disclosure provides a single device for measuring different parts of the body, which provides digital representation of results with improved resolution and accuracy.

The proposed disclosure provides a single device for measuring different parts of the body, which can transfer the measured data to various digital platforms for storage and analysis purpose.

We claim:

1. A device (100) for measuring handgrip strength and assessing muscle groups of a user, the device comprising:
a housing (102) adapted to be gripped in a hand of a user (302);
one or more load cells (104) enclosed inside the housing (102), wherein the one or more load cells (104) comprises a first load cell (104-1) and a second load cell (104-2) configured to support a predefined weight with an individual capacity, a third load cell (104-3) is configured to support a predefined weight pertaining to a finger of the user (302);
a finger flap (800) extending outwards when in use and unfolded back into the housing (102), wherein the finger flap (800) is configured to measure different types of finger strength, by holding the device (100) in any orientation,
wherein when the finger flap (800) is open, the third load cell (104-3) gets activated automatically and the rest of the load cells (104-1, 104-2) get deactivated at this point so one can easily hold the device from anywhere,
wherein the housing (102) configured to translate a force applied on the housing (102) to the one or more load cells (104), wherein the one or more load cells (104) are configured to generate a first set of electrical signals corresponding to the force applied on the housing (102);
an amplifier (106) operatively coupled to the one or more load cells (104), the amplifier (106) configured to amplify an amplitude of the first set of electrical signals to a predefined amplitude;
an analog to digital converter (ADC) module (108) operatively coupled to the amplifier (106), the ADC module (108) configured to receive the amplified first set of electrical signals and convert it into a first set of digital signals; and
a computing unit (110) operatively coupled to the ADC module (108), the computing unit (110) comprising one or more processors coupled with a memory, the memory storing instructions executable by the one or more processors to:
receive the first set of digital signals from the ADC module (108) and process the received first set of digital signals to generate a second set of digital signals, wherein the second set of digital signals corresponds to the force applied on the housing of the device (100),
wherein the generated second set of digital signals is processed by the computing unit (110) so as to be represented on at least one mobile computing device (202).

2. The device (100) as claimed in claim 1, wherein the one or more load cells (104) are a bending load cells, and the housing configured to translate the force applied on the housing by the user (302), into a bending force, to the bending load cells.

3. The device (100) as claimed in claim 1, wherein the predefined weight supported by the first load cell (104-1) and the second load cell (104-2) is 1 kilogram force to 70 kilograms force, wherein the predefined weight supported by the third load cell (104-3) is 1 kilogram force to 10 kilograms force.

4. The device (100) as claimed in claim 1, wherein the device (100) is configured to:
assess one or more muscle groups of the user based on a perpendicular position, an open position and a closed position of a set of handles (204) at a predefined angle, wherein the set of handles (204) measures one or more movements of a neck, a back, an arm and a hand,
wherein the predefined angle of the perpendicular position, the open position and the closed position pertains to Odegree, 90 degree and 180 degrees, respectively.

5. The device (100) as claimed in claim 1, wherein the device (100) is used in one or more posture of the user, wherein the one or more posture comprises at least one of a supine lying posture, a side lying posture, a prone lying posture, a sitting posture and a standing posture.

6. The device (100) as claimed in claim 1, wherein the one or more load cells (104) comprises one or more resistors configured to change resistance based on the bending force of the device (100).

7. The device (100) as claimed in claim 1, wherein the device (100) comprises:
a communication module (112) operatively coupled to the computing unit (202), and wherein the communication module (112) is configured to communicatively couple the device (100) to the at least one mobile computing device (202) and transmit the second set of digital signals corresponding to the force applied on the housing to the at least one mobile computing devices (202) of users.

8. The device (100) as claimed in claim 1, wherein the device (100) comprises a display module (114) operatively coupled to the computing unit (202), and
wherein the generated set of second signals corresponding to the force applied on the housing of the device is processed by the computing unit (202) so as to be represented on the display module (114).
